(19) United States
(12) Patent Application Publication
(10) Pub. No.: US 2013/0111968 A1
(43) Pub. Date: May 9, 2013

(54) SAMPLE ANALYSIS DEVICE FOR DETERMINING SAMPLES IN A SAMPLE MATRIX AND METHOD FOR DETERMINING SAMPLES IN ONE OR SEVERAL MATRICES

(71) Applicant: SERAMUN DIAGNOSTICA GMBH, Heidesee OT Wolzig (DE)

(72) Inventors: Tomas Porstmann, Heidesee OT Wolzig (DE); Klemens Loster, Hohen Neuendorf OT Bergfelde (DE); Erik Hochel, Berlin (DE); Christoph Lorenz, Wiesbaden (DE); Peter Pas, Berlin (DE)

(73) Assignee: SERAMUN DIAGNOSTICA GMBH, Heidesee OT Wolzig (DE)

(21) Appl. No.: 13/667,033

(22) Filed: Nov. 2, 2012

(30) Foreign Application Priority Data
Nov. 4, 2011 (DE) 102011055070.4

(51) Int. Cl. G01N 33/00; G01N 21/00

(52) U.S. Cl. 73/1.02; 73/61.41; 356/72

(57) ABSTRACT

A sample analysis device is provided for determining samples in a sample matrix with several sample volumes. The device includes: a receiving device with a receiving facility for a sample matrix comprising a plurality of sample volumes; a first measuring device configured to determine samples in the sample matrix, by means of wet measuring; a second measuring device, configured to determine further samples in the sample matrix, which further samples differ from the samples, or in another sample matrix, by means of dry measuring; and a transfer device, which is coupled to the receiving device and is configured to transfer the receiving device between a loading position and a plurality of measuring positions in such a manner that the receiving facility can be transferred to various measuring positions that are either associated with the first or with the second measuring device.

### CROSS-REFERENCE TO RELATED APPLICATIONS

This application claims the benefit and priority of German Application No. 10 2011 055 070.4, filed Nov. 4, 2011. The entire disclosure of the above application is incorporated herein by reference.

### FIELD

The present disclosure relates to a sample analysis device for determining samples in a sample matrix with several sample volumes, and to a method for determining samples in one or several sample matrices, each comprising several sample volumes.

### BACKGROUND

The statements in this section merely provide background information related to the present disclosure and may not constitute prior art.

Such technologies are used to determined substances to be investigated, for example bodily fluids, wherein for the purpose of analysis the substances to be investigated are placed in an arrangement comprising several sample volumes for receiving the substances to be determined, for example in the form of wells. The substance samples received in the sample matrix can then be determined with the use of various analysis methods, for example for medical diagnostic purposes.

The microtiter plate, for example comprising 96 or 384 sample volumes, is one known form of such sample matrices. It frequently comprises a plastic material. A matrix-like arrangement of sample volumes or wells is formed, each being suitable for separately receiving a substance sample. In this arrangement, several samples of one and the same substance to be determined, or samples of different substances, can be received in a microtiter plate.

Subsequently, the sample matrix is examined by means of analysis devices. From document DE 20 2008 009 859 U1 a device is known that is used to spectroscopically examine such a sample matrix. The luminescence radiation and/or the fluorescence radiation can be used as measuring quantities. In document WO 01/04608 A1, too, sample matrices are visually examined.

Furthermore, document DE 37 36 632 C2 relates to a device for analysing samples in a sample matrix. In one embodiment of the device the latter comprises two processing lines, which makes it possible to carry out two different measuring processes at the same time.

From document EP 0 569 215 A2 an analysis device is known in which a sample is examined by means of one or several measuring methods which in turn are selected from several different measuring methods that can be carried out with the same measuring device.

In document U.S. Pat. No. 4,528,159 A an automatic instrument system for analysing a patient sample is disclosed.

There is need for an improved sample analysis device for determining samples in a sample matrix and a method for determining samples in one or several sample matrices, by means of which device and method the analysis of a multitude of individual samples of one or several substances in a sample matrix, which substances are to be analysed, can be carried out more efficiently and with less effort and expenditure to the user.

### SUMMARY

This section provides a general summary of the disclosure, and is not a comprehensive disclosure of its full scope or all of its features.

According to one aspect, the disclosure relates to a sample analysis device for determining samples in a sample matrix with several sample volumes. The sample analysis device may comprise: a receiving device with a receiving facility for a sample or probe matrix comprising a plurality of sample or probe volumes; a first measuring device configured to determine samples in the sample matrix, when the sample matrix is arranged in the receiving facility, by means of wet measuring; a second measuring device, configured to determine further samples in the sample matrix, which further samples differ from the samples, or in another sample matrix, when the other sample matrix is arranged in the receiving facility, by means of dry measuring, and a transfer or relocation device which is coupled to the receiving device and is configured to transfer or relocate the receiving device between a loading position and a plurality of measuring positions in such a manner that the receiving facility can be transferred or relocated to various measuring positions that are either associated with or assigned to the first or with the second measuring device.

In another aspect, the disclosure relates to a method for determining samples in one or several sample matrices, each comprising several sample volumes, in a sample analysis device. The method comprising: loading a receiving device with a receiving facility in a loading position with a sample matrix comprising a plurality of sample volumes; transferring the receiving device with the sample matrix in the receiving facility by means of a transfer device to first measuring positions that are associated with a first measuring device, determining samples in the sample matrix in a wet measuring or humid measuring process by means of the first measuring device in the first measuring positions, transferring the receiving device with the sample matrix in the receiving facility by means of the transfer device to second measuring positions that are associated with a second measuring device that differs from the first measuring device, and determining further samples, which differ from the samples, in the sample matrix or in another sample matrix, arranged subsequently and optionally instead of the sample matrix in the receiving facility, in a dry measuring process by means of the second measuring device in the second measuring positions.

The sample matrix may be provided by a microtiter plate.

Sample matrices comprising a flat arrangement of several sample volumes, which sample volumes can in each case individually, i.e. separately of each other, may receive a substance to be determined. The substances to be determined may be regularly analytes in fluids, for example bodily fluids. After placement of the fluids into the sample volume and/or into the sample volumes, said fluids may be subject to solid-phase separation, and the analytes to be determined may be treated, for example in that a colour reaction is triggered. For example, treatment can take place by means of an enzyme-marked detection system, and a solid-phase immuno assay or a solid-phase hybridisation assay can be carried out.

Depending on the analysis or analyses carried out in relation to the samples to be determined, the sample matrix may comprise wet samples or humid samples and/or dry samples. The wet samples or humid samples may be characterised in that a fluid sample is arranged in the associated sample volume. In contrast to this, the sample volume of a dry sample is dry, in other words free of any fluid. In this process the sample volume was dried prior to the impending sample determination, for example after an analysis reaction. In the dried sample volume an analysable result of the analysis reaction remains, for example in the form of one or several dyed measuring fields or analysis fields at the bottom of the sample volume.

In one and the same sample analysis device, both wet samples or humid samples and dry samples may be determined by means of wet measuring or humid measuring with the use of a first measuring device and by means of dry measuring with the use of a second measuring device that differs from the first measuring device. In order to carry out the different measuring processes, the sample matrix may be moved to measuring positions that in each case are associated either with the first measuring device or with the second measuring device in such a manner that these are positions of the receiving facility with the sample matrix, in which positions one or several desired sample volumes can then be measured with the respective measuring device. In this arrangement it can be provided that in relation to a sample matrix received in the receiving facility of the receiving device exclusively wet measuring or humid measuring or exclusively dry measuring be carried out. However, it is also possible to provide for the use of both wet measuring or humid measuring and dry measuring in respect of one and the same sample matrix. In this context a preferred embodiment provides that within the framework of carrying out a first measuring type, i.e. either wet measuring/humid measuring or dry measuring, during evaluation of the measuring results it is checked whether in relation to the same sample matrix carrying out the second measuring type is also necessary. For example, the measuring results of wet measuring or humid measuring can be evaluated as to whether adequate measuring accuracy has been provided. If this is not the case, dry measuring can be carried out in addition. In this manner it would, for example, be possible to carry out a measuring regime that, depending on the result of a first measuring type, for the same or different substances optionally uses the second measuring type for determination.

It can be provided for the sample analysis device to comprise an adjustment mechanism for the various measuring positions, which may be implemented by means of suitable hardware components and/or software components. In this process the receiving facility with the sample matrix may be first, by means of the transfer device, brought to a measuring position region of the respective measuring device. In order to then bring the sample volume to be examined at the time into the correct measuring position, the adjustment mechanism may check whether the correct measuring position has already been assumed. This can, for example, be carried out in that a photometric record of the sample volume is made by means of a camera, and the record is evaluated by means of image analysis. Thus it is possible in this manner, for example, to check the position of measuring regions or measuring fields at the bottom of the sample volume and/or of an adjustment aid at the sample volume in order to subsequently, by means of the adjustment mechanism, if appropriate carry out readjustments in order to assume the correct measuring position in relation to the associated measuring device; for which purpose preferably the transfer device is then selected.

It may be provided for the first and/or the second measuring device to be designed as an optical measuring device. By means of the measuring devices it may be possible, for example, to use the following optical measuring methods: absorption measuring, fluorescence measuring, phosphorescence measuring and/or extinction measuring. Furthermore, in one embodiment the dynamic behaviour of the examined sample can be examined, for example by means of measuring a time-dependent change in extinction or a fluorescence decay time. In one embodiment of the sample analysis device during wet measuring or humid measuring the extinction/absorption of the wet sample or humid sample in transmitted light is measured. To this effect the actually examined sample volume is illuminated from one side with a measuring light beam. On the opposite side by means of a photo detector, the light transiting through the sample volume is then acquired in order to examine it, for example in a spectrum-dependent manner and/or in regard to the light intensity passing through. In this embodiment, dry measuring is carried out as photometric measuring in such a manner that a visual image is acquired of the sample volume, in particular the bottom of the sample volume, for example by means of a camera, which image is subsequently fed for software-based image analysis. In this manner, for example, different colouration of measuring spots or measuring fields at the bottom of the dried sample volume is possible. In conjunction with the different measuring methods it can be provided for the light radiated onto the sample volume that is to be examined to be made available by means of one or several light-emitting diodes (LEDs). In conjunction with photometric measuring it is possible, in this manner, to use, for example, an LED matrix in order to produce as uniform as possible an illumination of the sample volume for the image acquisition.

It may be provided for the transfer device to be configured, within a matrix arrangement of sample volumes, which matrix arrangement is formed in the sample matrix and/or in the other sample matrix, in respect of a first group of sample volumes to move to measuring positions that are associated with wet measuring or humid measuring with the first measuring device, and in respect of a second group of sample volumes, which group differs from the first group of sample volumes, to move to other measuring positions that are associated with dry measuring using the second measuring device. In this embodiment, for determining samples within a matrix arrangement with n×m (n, m>0) sample volumes, both wet measuring or humid measuring and dry measuring are used, in that in each case movement to the associated measuring positions takes place by means of the transfer device. In the simplest case it can also be a matrix arrangement comprising precisely two sample volumes, of which one sample volume is determined by means of the first measuring device within the framework of wet measuring or humid measuring, while the other sample volume is determined by means of the second measuring device within the framework of dry measuring. However, usually the matrix arrangement will comprise a larger number of sample volumes arranged in a matrix-like manner. This matrix arrangement then comprises a first group of sample volumes, namely wet samples or humid samples, and a second group, which differs from said first group, of sample volumes, which are dry samples. The different sample volumes are then determined by means of the measuring device that is in each case associated with the sample type.

The transfer device may comprise an x-y transfer unit.

A readout device may be provided which may be configured to read out usage information from an information storage device on the sample matrix in a non-contacting manner. The information storage device on the sample matrix can, for example, be an optically readable barcode or a radio frequency identification (RFID) transponder. Likewise, a combination of such information storage devices can be provided. By means of the information storage device it is possible to provide any desired usage information for non-contacting read-out in the sample analysis device.

A checking device may be provided that may be functionally coupled to the readout device and that is configured to evaluate the read-out usage information in such a manner that determining samples of the sample matrix is prevented if during evaluation of the usage information it is detected that the sample matrix is not of an approved design. This ensures that the sample analysis device is operated exclusively with sample matrices approved for this purpose. This prevents, for example, the sample analysis device from being damaged because of the use of non-approved sample matrices. It also ensures that the measuring results are not compromised due to the use of non-approved sample matrices.

A measuring control device may be provided that may be functionally coupled to the readout device and to the first and/or second measuring device and that is configured to evaluate the read-out usage information in such a manner that determining the samples in the sample matrix by means of the first and/or the second measuring device is carried out according to measuring regime information that is derived, by the measuring control device, from the read-out usage information. In this embodiment the usage information comprises information as to which sample volume on the sample matrix is associated with which measuring device. This can be provided in respect of one or several of the sample volumes on the matrix. The measuring control device, optionally in cooperation with the control device of the analysis device, during evaluation of this usage information then, by means of corresponding control signals, ensures that the respective sample volume moves to the associated measuring position by means of the transfer device, and subsequently wet measuring or humid measuring or dry measuring is carried out. The usage information thus determines which sample volume (well) of the sample matrix is fed to which measuring process. Furthermore, it can be provided for a sequence to be derived from the usage information, in which sequence the sample volumes are fed to the first and/or to the second measuring device.

A measuring checking device may be provided that may be functionally coupled to the first and/or the second measuring device and that is configured, prior to, during, and/or after wet measuring or humid measuring with the first measuring device and/or dry measuring with the second measuring device, to check whether an actually determined sample is approved for the respective measuring type. In order to prevent, for example, determining a dry sample by means of wet measuring or humid measuring with the first measuring device, this embodiment provides for a measuring checking device which in the context of a carried-out measuring process checks whether the actually examined sample is determined with the correct measuring type. For example an image acquisition of the sample volume can be analysed as to whether expected measuring fields on the bottom of the sample volume can in principle be acquired. If this is not the case the reason for this may, for example, be that it is a wet sample or humid sample, in which thus a fluid is arranged in the sample volume.

In conjunction with the method for determining samples in one or several sample matrices the embodiments described above in the context of the sample analysis device may be provided accordingly.

In the method for determining samples, it may be provided that during wet measuring or humid measuring by means of the first measuring device and/or during dry measuring by means of the second measuring device at least one analysis parameter from the following group is determined: autoantibodies, antibodies against infectious agents, and antigens of infectious agents. It is possible, in particular, to determine parameters in conjunction with solid-phase enzyme immune techniques or solid-phase hybridisation techniques.

### BRIEF DESCRIPTION OF THE DRAWINGS

The present disclosure will become more fully understood from the detailed description and the accompanying drawings, wherein:

FIG. 1 is a diagrammatic view of a sample analysis device for determining samples in a sample matrix with several sample volumes, and

FIG. 2 is an image relating to a dry sample.

### DETAILED DESCRIPTION

The following description is merely exemplary in nature and is not intended to limit the present disclosure, application, or uses. It should be understood that throughout the drawings, corresponding reference numerals indicate like or corresponding parts and features.

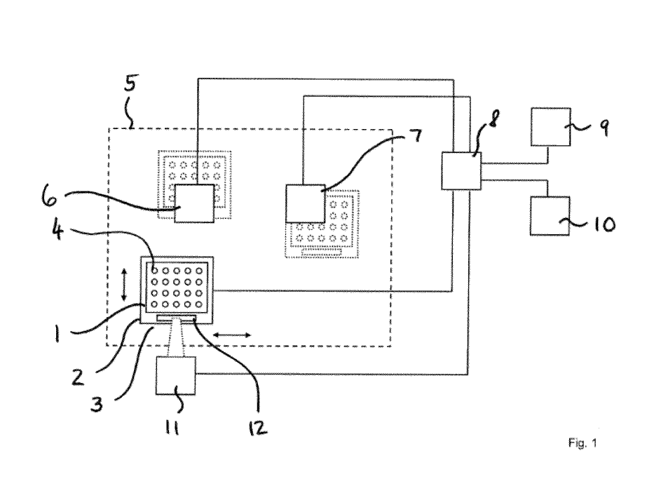

FIG. 1 shows a diagrammatic view of a sample analysis device in which a sample matrix 1 is arranged in a receiving facility 2 of a transfer device 3. By means of the transfer device 3 the sample matrix 1, which comprises an arrangement of sample volumes 4, can be transferred to a transfer region 5. In this manner the sample matrix 1 can be moved between a loading position, shown in FIG. 1, and several positions in the transfer region so that, in particular, measuring positions can be assumed in which the sample matrix 1 is arranged in positions that are associated with a first or a second measuring device 6, 7, which in FIG. 1 is shown by means of dashed lines.

The sample analysis device shown in FIG. 1 further comprises a control device 8 which is coupled not only to the two measuring devices 6, 7, but also to a display device 9 and to an input device 10, which is, for example, an input keyboard or an input field. Further system components can be coupled to the control device and/or to the other devices, for example a data interface (not shown).

Furthermore, the control device 8 according to FIG. 1 is coupled to the transfer device 3 and to a readout device 11. By means of the readout device 11 a storage device 12 arranged on the sample matrix 1 can be read-out in a non-contacting manner, for example a barcode by means of optical readout. In an alternative embodiment the storage device 12 can comprise an RFID chip.

Usage information read-out in this manner can then be evaluated in the control device 9 for measuring operation. For example, with reference to the read-out usage information it is possible to check whether the sample matrix 1 is a sample matrix that is approved for the sample analysis device. If this is not the case, any further handling of the inserted sample matrix 1 in the sample analysis device is blocked by the control device 8. In this context it can then be provided for a warning alert or an information note to be issued, by the control device 8, to the user by way of the display device 9.

Furthermore, in conjunction with the usage information it can be provided for the aforesaid to comprise measuring information that shows which of the sample volumes 4 (wells) of the sample matrix 1 are provided for a particular type of analysis or measuring process. Thus the usage information can state that a first row or column of the matrix of sample volumes 4 is associated with the first measuring device 6, whereas a second row or column of the sample volumes 4 is to be evaluated with the second measuring device 7. This information can then be used by the control device 8 in order to control the transfer device 3 and the measuring devices 6, 7 during determination of the samples that are received in the sample matrix 1.

By means of software components and hardware components which optionally can at least in part be integrated in the control device 8, moreover further functional devices or functional components of the sample analysis device can be implemented. This includes, for example, an adjustment application to the effect that, for example, fine adjustment of the sample matrix 1 is carried out in the measuring position in relation to the respective measuring device.

In the embodiment shown the first measuring device 6 is a measuring device that is configured to carry out an extinction measuring process and/or an absorption measuring process in relation to the sample to be determined. To this effect a measuring light beam is directed onto the sample to be examined, which sample is a wet sample or a humid sample, onto the corresponding sample volume, and subsequently the light that has gone through the sample is acquired by means of a photo detector in order to evaluate it, for example in terms of its spectral distribution and/or its intensity when compared to the beamed-in measuring light beam. Thus by means of the first measuring device 6 a wet measuring process or humid measuring process for a wet sample or a humid sample can be carried out, which is characterised in that in the associated sample volumes 4 a fluid sample of the substance to be analysed is arranged.

In contrast to this, the second measuring device 7 in the exemplary embodiment shown is equipped to carry out a dry measuring process for a dry sample in which the sample volume 4 to be examined has been dried again after one or several analysis reactions were carried out in respect of the substance to be analysed. The dry measuring process can, for example, comprise an image acquisition of the bottom of the examined sample volume 4.

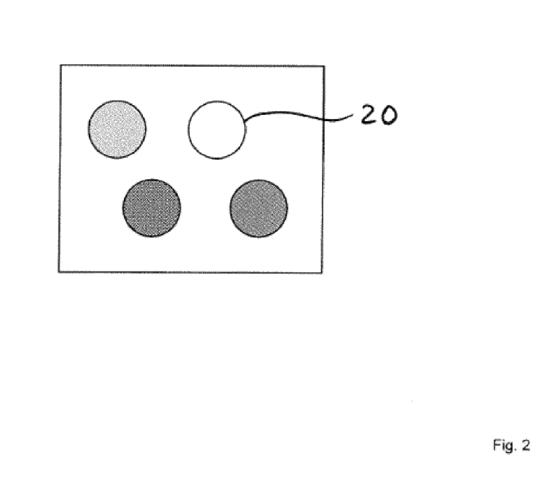

FIG. 2 shows a diagrammatic view of such an image with several measuring regions 20 which due to different analysis reactions are different in colour. The image can then, by means of evaluation software, be subjected to image analysis and finally determination of the sample can be carried out. In a special embodiment the image analysis takes place following previous adjustment of the image by means of coloured measuring regions or by means of register marks or other marks on the sample volumes of the sample matrices.

It will be appreciated that while various embodiments have been described, those skilled in the art will recognize modifications or variations which might be made without departing from the present disclosure. Thus, the present examples are not intended to limit the present disclosure. Therefore, the description and claims should be interpreted liberally with only such limitation as is necessary in view of the pertinent prior art.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A sample analysis device for determining samples in a sample matrix with several sample volumes, comprising:
a receiving device with a receiving facility for a sample matrix comprising a plurality of sample volumes;
a first measuring device configured to determine samples in the sample matrix, when the sample matrix is arranged in the receiving facility, by wet measuring;
a second measuring device, configured to determine further samples in the sample matrix, which further samples differ from the samples, or in another sample matrix, when the other sample matrix is arranged in the receiving facility, by dry measuring; and
a transfer device which is coupled to the receiving device and is configured to transfer the receiving device between a loading position and a plurality of measuring positions in such a manner that the receiving facility can be transferred to various measuring positions that are either associated with the first or with the second measuring device.

2. The sample analysis device according to claim 1, wherein at least one of the first and the second measuring device are designed as an optical measuring device.

3. The sample analysis device according to claim 1, wherein the transfer device is configured, within a matrix arrangement of sample volumes, which matrix arrangement is formed in at least one of the sample matrix and in the other sample matrix, in respect of a first group of sample volumes to move to measuring positions that are associated with wet measuring or humid measuring with the first measuring device, and in respect of a second group of sample volumes, which group differs from the first group of sample volumes, to move to other measuring positions, which are associated with dry measuring using the second measuring device.

4. The sample analysis device according to claim 1, wherein the transfer device comprises an x-y transfer unit.

5. The sample analysis device according to claim 1, wherein a readout device that is configured to read out usage information from an information storage device on the sample matrix in a non-contacting manner.

6. The sample analysis device according to claim 5, wherein a checking device is provided that is functionally coupled to the readout device and that is configured to evaluate the read-out usage information in such a manner that determining samples of the sample matrix is prevented if during evaluation of the usage information it is detected that the sample matrix is not of an approved design.

7. The sample analysis device according to at least one of claim 5 or 6, wherein a measuring control device is provided that is functionally coupled to the readout device and to at least one of the first and the second measuring device and that is configured to evaluate the read-out usage information in such a manner that determining the samples in the sample matrix by means of at least one of the first and the second measuring device is carried out according to measuring regime information that is derived, by the measuring control device, from the read-out usage information.

8. The sample analysis device according to claim 1, wherein a measuring checking device is provided that is functionally connected to at least one of the first and the second measuring device and that is configured prior to, during or after wet measuring or humid measuring with the first measuring device or dry measuring with the second measuring device, to check whether an actually determined sample is approved for the respective measuring type.

9. A method for determining samples in one or several sample matrices, each comprising several sample volumes, in a sample analysis device, the method comprising:
loading a receiving device with a receiving facility in a loading position with a sample matrix comprising a plurality of sample volumes,
transferring the receiving device with the sample matrix in the receiving facility by means of a transfer device to first measuring positions that are associated with a first measuring device,
determining samples in the sample matrix in a wet measuring or humid measuring process by means of the first measuring device in the first measuring positions,
transferring the receiving device with the sample matrix in the receiving facility by means of the transfer device to second measuring positions that are associated with a second measuring device that differs from the first measuring device, and
determining further samples, which differ from the samples, in the sample matrix or in another sample matrix, arranged subsequently and optionally instead of the sample matrix in the receiving facility, in a dry measuring process by means of the second measuring device in the second measuring positions.

10. The method according to claim 9, wherein during wet measuring or humid measuring by means of the first measuring device or during dry measuring by means of the second measuring device at least one analysis parameter is determined from the group consisting of: autoantibodies, antibodies against infectious agents, and antigens of infectious agents.

11. A sample analysis device for determining samples in a sample matrix with several sample volumes, comprising:
a receiving device with a receiving facility for a sample matrix comprising a plurality of sample volumes;
a first measuring device configured to determine samples in the sample matrix, when the sample matrix is arranged in the receiving facility, by means of wet measuring;
a second measuring device, configured to determine further samples in the sample matrix, which further samples differ from the samples, or in another sample matrix, when the other sample matrix is arranged in the receiving facility, by means of dry measuring; and
a transfer device which is coupled to the receiving device and is configured to transfer the receiving device between a loading position and a plurality of measuring positions in such a manner that the receiving facility can be transferred to various measuring positions that are either associated with the first or with the second measuring device.
